(19) United States
(12) Patent Application Publication
(10) Pub. No.: US 2017/0022574 A1
(43) Pub. Date: Jan. 26, 2017

(54) MOLECULAR MARKERS ASSOCIATED WITH HAPLOID INDUCTION IN ZEA MAYS

(71) Applicants: Rahul Dhawan, St. Louis, MO (US); Jennifer L. Jacobs, St. Charles, MO (US); Warren M. Kruger, Glencoe, MO (US); Bryce M. Lemke, Nevada, IA (US); Ryan A. Rapp, Addieville, IL (US); Minghui Sun, Ames, IA (US); Christopher A. Taylor, Ballwin, MO (US)

(72) Inventors: Rahul Dhawan, St. Louis, MO (US); Jennifer L. Jacobs, St. Charles, MO (US); Warren M. Kruger, Glencoe, MO (US); Bryce M. Lemke, Nevada, IA (US); Ryan A. Rapp, Addieville, IL (US); Minghui Sun, Ames, IA (US); Christopher A. Taylor, Ballwin, MO (US)

(21) Appl. No.: 15/243,304

(22) Filed: Aug. 22, 2016

Related U.S. Application Data
(63) Continuation of application No. 13/819,490, filed on Aug. 7, 2013, now abandoned.
(60) Provisional application No. 61/450,300, filed on Mar. 8, 2011.
(60) Provisional application No. 61/378,674, filed on Aug. 31, 2010.

(51) Int. Cl. C12Q 1/68; A01H 1/04; A01H 5/10

(52) U.S. Cl. CPC C12Q 1/6895; A01H 5/10; A01H 1/04; C12Q 2600/156; C12Q 2600/13; C12Q 2600/158; C12Q 2600/172

(57) ABSTRACT

The present invention is the field of plant breeding. More specifically, the invention focuses on the use of molecular markers to select for a genetic locus contributing to haploid induction.

### CROSS-REFERENCE TO RELATED APPLICATIONS

This application claims priority from U.S. patent application Ser. No. 13/819,490 (filed Aug. 7, 2013), U.S. Provisional Application Ser. No. 61/378,674 filed (Aug. 31, 2010) and U.S. Provisional Application Ser. No. 61/450,300 filed (Mar. 8,2011), each of which are hereby incorporated herein in their entirety.

### INCORPORATION OF SEQUENCE LISTING

A sequence listing containing the file named “46_21_57579_1.txt” which is 26,539 bytes (measured in MS-Windows®) and created on 31 Aug. 2011, comprises 55 nucleotide sequences, and is herein incorporated by reference in its entirety.

### BACKGROUND

Plant breeding is greatly facilitated by the use of doubled haploid (DH) plants. The production of DH plants enables plant breeders to obtain inbred lines without multi-generational inbreeding, thus decreasing the time required to produce homozygous plants. DH plants provide an invaluable tool to plant breeders, particularly for generating inbred lines, QTL mapping, cytoplasmic conversions, and trait introgression. A great deal of time is spared as homozygous lines are essentially instantly generated, negating the need for multigenerational conventional inbreeding. In particular, because DH plants are entirely homozygous, they are very amenable to quantitative genetics studies. Both additive variance and additive crossed by additive genetic variances can be estimated from DH populations. Other applications of DH technology include identification of epistasis and linkage effects. Moreover, there is value in testing and evaluating homozygous lines for plant breeding programs. All of the genetic variance is among progeny in a breeding cross, which improves selection gain. Methods of utilizing haploids in genetic studies have been well described in the art. A statistical method to utilize pooled haploid DNA to estimate parental linkage phase and to construct genetic linkage maps has been described (Gasbarra, D. et al., Genetics 172:1325-1335 (2006). An additional study has used the method of crossing haploid wheat plants with cultivars to map leaf rust resistance gene in wheat (Hiebert, C. et al., Theor Appl Genet 110:1453-1457 (2005). Haploid plants and SSR markers have been used in linkage map construction of cotton (Song, X. et al., Genome 48:378-392 (2005). Furthermore, AFLP marker analysis has been performed in monoploid potato (Varrieur, 1., Thesis, AFLP Marker Analysis of Monoploid Potato (2002)

Haploids are traditionally generated through an androgenesis or gynogenesis approach (Hiebert, C. et al., Theor Appl Genet 117:581-594 (2008). In corn, the haploids are generated spontaneously when crossed to the maize inducer lines.

Breeding crop plants is greatly facilitated by the use of marker-assisted selection. Of the classes of genetic markers, single nucleotide polymorphisms (SNPs) have characteristics which make them preferential to other genetic markers in detecting, selecting for, and introgressing disease resistance in a com plant. SNPs are preferred because technologies are available for automated, high-throughput screening of SNP markers, which can decrease the time to select for and introgress disease resistance in com plants. Further, SNP markers are ideal because the likelihood that a particular SNP allele is derived from independent origins in the extant population of a particular species is very low. As such, SNP markers are useful for tracking and assisting introgression of disease resistance alleles, particularly in the case of disease resistance The present invention defines a novel haplotype, SNP markers associated with it and method of using this for predictive breeding and haploid identification.

### SUMMARY

The production of haploid seed is critical for the doubled haploid breeding process. Haploid seed are produced on maternal germplasm when fertilized with pollen from a gynogenetic inducer, such as Stock 6. The present invention identifies a locus that increases haploid induction frequency and use of molecular markers to support haploid identification. The locus was identified by comparing the genetic fingerprint data of a panel of gynogenetic inducer lines to elite germplasm. The locus is conserved amongst all inducer lines, but is not contained in elite inbred germplasm.

In one embodiment, the locus for increasing haploid induction frequency can be found on chromosome 1 in a genomic region flanked by or including a) loci NC0016876 and NC0039812; b) loci NZMAY008358670 and loci NC0039812; or c) loci NC0016876 and loci NZMAY008358232

In one embodiment, the invention is directed to a method for identifying a maize plant that comprises a genotype associated with an increased haploid induction phenotype. The method comprises detecting in a maize plant an allele in at least one haploid induction locus associated with an increased haploid induction phenotype wherein the haploid induction locus is on chromosome 1 in a genomic region flanked by or including a) loci NC0016876 and NC0039812; b) loci NZMAY008358670 and loci NC0039812; or c) loci NC0016876 and loci NZMAY008358232.

In another embodiment, the invention is directed to a method for obtaining a maize plant comprising in its genome at least one haploid induction locus. The method comprises genotyping a plurality of maize plants with respect to at least one haploid induction locus on chromosome 1 in a genomic region flanked by or including (a) loci NC0016876 and NC0039812; (b) loci NZMAY008358670 and loci NC0039812; or (c) loci NC0016876and loci NZMAY008358232; and selecting a maize plant comprising in its genome at least one haploid induction locus comprising a genotype associated with a increased haploid induction phenotype.

Further aspects and embodiments of the present invention will be apparent from the description provided herein. It should be understood that the description and examples provided are intended for purposes of illustration only and are not intended to limit the scope of Applicants' invention.

### DESCRIPTION

### I. Definitions

The definitions and methods provided herein define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et aI., Molecular Biology of The Cell, 3rd Edition, Garland Publishing, Inc.: New York, 1994; Rieger et aI., Glossary of Genetics: Classical and Molecular, 5th Edition, Springer-Verlag: New York, 1991; and Lewin, Genes V, Oxford University Press: New York, 1994. The nomenclature for DNA bases as set forth at 37 CFR §1.822 is used.

As used herein, a “locus” is a fixed position on a chromosome and may represent a single nucleotide, a few nucleotides or a large number of nucleotides in a genomic region.

As used herein, “polymorphism” means the presence of one or more variations of a nucleic acid sequence at one or more loci in a population of one or more individuals. The variation may comprise but is not limited to, one or more base changes, the insertion of one or more nucleotides or the deletion of one or more nucleotides. A polymorphism includes a single nucleotide polymorphism (SNP), a simple sequence repeat (SSR) and indels, which are insertions and deletions. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the later may be associated with rare but important phenotypic variation.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

As used herein, “genetic marker” means polymorphic nucleic acid sequence or nucleic acid feature. A “polymorphism” is a variation among individuals in sequence, particularly in DNA sequence, or feature, such as a transcriptional profile or methylation pattern. Useful polymorphisms include single nucleotide polymorphisms (SNPs), insertions or deletions in DNA sequence (Indels), simple sequence repeats of DNA sequence (SSRs) a restriction fragment length polymorphism, a haplotype, and a tag SNP. A genetic marker, a gene, a DNA-derived sequence, a RNA-derived sequence, a promoter, a 5′ untranslated region of a gene, a 3′ untranslated region of a gene, micro RNA, siRNA, a QTL, a satellite marker, a transgene, mRNA, ds mRNA, a transcriptional profile, and a methylation pattern may comprise polymorphisms.

As used herein, “marker assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), micro array-based technologies, and nucleic acid sequencing technologies, etc.

As used herein, the phrase “immediately adjacent”, when used to describe a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “immediately adjacent” to the polymorphism.

As used herein, “interrogation position” refers to a physical position on a solid support that can be queried to obtain genotyping data for one or more predetermined genomic polymorphisms.

As used herein, “consensus sequence” refers to a constructed DNA sequence which identifies SNP and Indel polymorphisms in alleles at a locus. Consensus sequence can be based on either strand of DNA at the locus and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

As used herein, the term “single nucleotide polymorphism,” also referred to by the abbreviation “SNP,” means a polymorphism at a single site wherein said polymorphism constitutes a single base pair change, an insertion of one or more base pairs, or a deletion of one or more base pairs.

As used herein, “genotype” means the genetic component of the phenotype and it can be indirectly characterized using markers or directly characterized by nucleic acid sequencing. Suitable markers include a phenotypic character, a metabolic profile, a genetic marker, or some other type of marker. A genotype may constitute an allele for at least one genetic marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome.

As used herein, the term “haplotype” means a chromosomal region within a haplotype window defined by at least one polymorphic molecular marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or trans gene. Any such change in a haplotype would be included in our definition of what constitutes a haplotype so long as the functional integrity of that genomic region is unchanged or improved.

As used herein, the term “haplotype window” means a chromosomal region that is established by statistical analyses known to those of skill in the art and is in linkage disequilibrium. Thus, identity by state between two inbred individuals (or two gametes) at one or more molecular marker loci located within this region is taken as evidence of identity-by-descent of the entire region. Each haplotype window includes at least one polymorphic molecular marker. Haplotype windows can be mapped along each chromosome in the genome. Haplotype windows are not fixed per se and, given the ever increasing density of molecular markers, this invention anticipates the number and size of haplotype windows to evolve, with the number of windows increasing and their respective sizes decreasing, thus resulting in an ever-increasing degree of confidence in ascertaining identity by descent based on the identity by state at the marker loci.

As used herein, a plant referred to as “haploid” has a single set (genome) of chromosomes and the reduced number of chromosomes (n) in the haploid plant is equal to that of the gamete.

As used herein, a plant referred to as “doubled haploid” is developed by doubling the haploid set of chromosomes. A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric.

As used herein, a plant referred to as “diploid” has two sets (genomes) of chromosomes and the chromosome number (2n) is equal to that of the zygote.

As used herein, the term “plant” includes whole plants, plant organs (Le., leaves, stems, roots, etc.), seeds, and plant cells and progeny of the same. “Plant cell” includes without limitation seeds, suspension cultures, embryos, meristematic regions, callus tissue, leaves, shoots, gametophytes, sporophytes, pollen, and microspores.

As used herein, a “genetic map” is the ordered list of loci known for a particular genome.

As used herein, “phenotype” means the detectable characteristics of a cell or organism which are a manifestation of gene expression.

As used herein, a “phenotypic marker” refers to a marker that can be used to discriminate phenotypes displayed by organisms.

As used herein, “linkage” refers to relative frequency at which types of gametes are produced in a cross. For example, iflocus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage V4 of the gametes will of each genotype. Segregation of gametes into a genotypes differing from V4 are attributed to linkage.

As used herein, “linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. Two loci are said to be “genetically linked” when they are in linkage disequilibrium. As used herein, “quantitative trait locus (QTL)” means a locus that controls to some degree numerically representable traits that are usually continuously distributed.

As used herein, the term “transgene” means nucleic acid molecules in form of DNA, such as cDNA or genomic DNA, and RNA, such as mRNA or micro RNA, which may be single or double stranded.

As used herein, the term “inbred” means a line that has been bred for genetic homogeneity.

As used herein, “linkage block” means a chromosomal region that is established by statistical analyses known to those of skill in the art and is in linkage disequilibrium. Thus, identity by state between two inbred individuals (or two gametes) at one or more loci located within this region is taken as evidence of identity-by-descent of the entire region. Linkage blocks can be mapped along each chromosome in the genome.

As used herein, the term “hybrid” means a progeny of mating between at least two genetically dissimilar parents. Without limitation, examples ‘of mating schemes include single crosses, modified single cross, double modified single cross, three-way cross, modified three-way cross, and double cross wherein at least one parent in a modified cross is the progeny of a cross between sister lines.

As used herein, the term “tester” means a line used in a testcross with another line wherein the tester and the lines tested are from different germplasm pools. A tester may be isogenic or nonisogenic. As used herein, “resistance allele” means the isolated nucleic acid sequence that includes the polymorphic allele associated with resistance to the disease or condition of concern.

As used herein, the term “com” means Zea mays or maize and includes all plant varieties that can be bred with com, including wild maize species.

As used herein, the term “com” means Zea mays or maize and includes all plant varieties that can be bred with com, including wild maize species.

As used herein, an “elite line” is any line that has resulted from breeding and selection for superior agronomic performance.

As used herein, an “elite line” is any line that has resulted from breeding and selection for superior agronomic performance. As used herein, an “inducer” is a line which is crossed with another line and promotes the formation of haploid embryos.

As used herein, “haplotype effect estimate” means a predicted effect estimate for a haplotype reflecting association with one or more phenotypic traits, wherein the associations can be made de novo or by leveraging historical haplotype-trait association data.

As used herein, “breeding value” means a calculation based on nucleic acid sequence effect estimates and nucleic acid sequence frequency values, the breeding value of a specific nucleic acid sequence relative to other nucleic acid sequences at the same locus (i.e., haplotype window), or across loci (i.e., haplotype windows), can also be determined. In other words, the change in population mean by fixing said nucleic acid sequence is determined. In addition, in the context of evaluating the effect of substituting a specific region in the genome, either by introgression or a transgenic event, breeding values provide the basis for comparing specific nucleic acid sequences for substitution effects. Also, in hybrid crops, the breeding value of nucleic acid sequences can be calculated in the context of the nucleic acid sequence in the tester used to produce the hybrid.

To the extent to which any of the preceding definitions is inconsistent with definitions provided in any patent or non-patent reference incorporated herein or in any reference found elsewhere, it is understood that the preceding definition will be used herein.

### II. Detailed Description: Overview

In accordance with the present invention, Applicants have discovered genomic regions, associated markers, and associated methods for identifying and associating genotypes that affect haploid induction. For example, in one embodiment, a method of the invention comprises screening germplasm for an increased haploid induction phenotype with the use of molecular markers.

Provided herein is a maize genomic region that is shown herein to be associated with a desirable haploid induction phenotype when present in certain allelic forms. A maize genomic region provided that can be associated with haploid induction when present in certain allelic forms is located on chromosome 1.

III. A Genomic Region Associated with Haploid Induction

TABLE 1
Markers spanning a genomic region associated with
haploid induction on chromosome 1 of Zea mays.

| | | | | | | | Allelic |
|---|---|---|---|---|---|---|---|
| | | IBM2 | | | | | Homozygous |
| | | Neighbors | | | | | form(s) |
| | Map | 2008 | | | | | Associated |
| Marker or Locus | Position | Genetic | SEQ | SNP | Start | End | with Haploid |
| Name | (cM) | Map¹ | ID: | Position | Position | Position | induction ² |
| NZMAY008359207 | 90.1 | | 1 | 101 | 48,249,509 | 48,249,309 | GG |
| NZMAY008359058 | 90.1 | | 2 | 101 | 48,413,950 | 48,413,750 | CC |
| NZMAY008359056 | 90.1 | | 3 | 101 | 48,413,999 | 48,413,799 | TT |
| NZMAY008359054 | 90.1 | | 4 | 101 | 48,414,070 | 48,413,870 | CC |
| NZMAY008359052 | 90.1 | | 5 | 101 | 48,414,197 | 48,413,997 | AA |
| NZMAY008359050 | 90.1 | | 6 | 101 | 48,414,355 | 48,414,155 | GG |
| NZMAY008359049 | 90.1 | | 7 | 101 | 48,414,422 | 48,414,222 | TT |
| NZMAY008359037 | 90.2 | | 8 | 101 | 48,415,754 | 48,415,554 | GG |
| NZMAY008359038 | 90.2 | | 9 | 101 | 48,416,195 | 48,415,995 | AA |
| NZMAY008359039 | 90.2 | | 10 | 101 | 48,416,450 | 48,416,250 | AA |
| NZMAY008359034 | 90.2 | | 11 | 101 | 48,416,743 | 48,416,543 | GG |
| NZMAY008359033 | 90.2 | | 12 | 101 | 48,416,746 | 48,416,546 | AA |
| NZMAY008359031 | 90.2 | | 13 | 101 | 48,416,774 | 48,416,574 | AA |
| NZMAY008359032 | 90.2 | | 14 | 101 | 48,416,823 | 48,416,623 | TT |
| NZMAY008359030 | 90.2 | | 15 | 101 | 48,416,941 | 48,416,741 | CC |
| NZMAY008358992 | 90.5 | | 16 | 101 | 48,366,021 | 48,365,821 | CC |
| NZMAY008358990 | 90.5 | | 17 | 101 | 48,366,369 | 48,366,169 | CC |
| NZMAY008358670 | 91.1 | | 18 | 101 | 49,170,593 | 49,170,793 | CC |
| NZMAY008358452 | 91.1 | | 19 | 101 | 49,287,620 | 49,287,420 | AA |
| NZMAY008358438 | 91.1 | | 20 | 101 | 49,298,534 | 49,298,334 | CC |
| NZMAY008358437 | 91.1 | | 21 | 101 | 49,298,755 | 49,298,555 | TT |
| NZMAY008358436 | 91.1 | | 22 | 101 | 49,298,762 | 49,298,562 | CC |
| NZMAY008358433 | 91.1 | | 23 | 101 | 49,298,842 | 49,298,642 | TT |
| NZMAY008358038 | 91.1 | | 24 | 101 | 49,917,388 | 49,917,188 | GG |
| NZMAY008357921 | 91.1 | | 25 | 101 | 49,979,002 | 49,978,802 | TT |
| NZMAY008358232 | 91.6 | | 26 | 101 | 49,530,002 | 49,530,202 | TT |
| NZMAY008358233 | 91.6 | | 27 | 101 | 49,530,342 | 49,530,542 | CC |
| NZMAY008358234 | 91.6 | | 28 | 101 | 49,530,365 | 49,530,565 | TT |
| NZMAY008358235 | 91.6 | | 29 | 101 | 49,530,373 | 49,530,573 | GG |
| NZMAY008358240 | 91.6 | | 30 | 101 | 49,531,530 | 49,531,730 | AA |
| NZMAY008358255 | 91.6 | | 31 | 101 | 49,535,291 | 49,535,491 | GG |
| NZMAY008357271 | 92.5 | | 32 | 101 | 50,887,856 | 50,887,656 | CC |
| NZMAY008357270 | 92.5 | | 33 | 101 | 50,887,876 | 50,887,676 | GG |
| NZMAY008357268 | 92.5 | | 34 | 101 | 50,887,943 | 50,887,743 | TT |
| NZMAY008357158 | 92.6 | | 35 | 101 | 50,894,262 | 50,894,062 | CC |
| NZMAY008356719 | 93.3 | | 36 | 101 | 51,199,249 | 51,199,049 | |
| NZMAY008356706 | 93.3 | | 37 | 101 | 51,202,029 | 51,201,829 | |
| NZMAY008356611 | 94.2 | | 38 | 101 | 51,883,139 | 51,882,939 | |
| NZMAY008361868 | 94.5 | | 39 | 101 | 53,296,765 | 53,296,965 | |
| NZMAY008361867 | 94.5 | | 40 | 101 | 53,296,724 | 53,296,924 | |
| NZMAY008359413 | 95 | | 41 | 101 | 52,640,679 | 52,640,879 | |
| NZMAY008359408 | 95.1 | | 42 | 101 | 52,640,107 | 52,640,307 | |
| NZMAY008359407 | 95.1 | | 43 | 101 | 52,640,205 | 52,640,405 | |
| NZMAY008359409 | 95.1 | | 44 | 101 | 52,640,351 | 52,640,551 | |
| NZMAY008359410 | 95.1 | | 45 | 101 | 52,640,462 | 52,640,662 | |
| NZMAY008359405 | 95.2 | | 46 | 101 | 52,639,659 | 52,639,859 | |
| NC0000509 | 100.7 | | 47 | 178 | | | AA or TT |
| NC0004387 | 99.1 | | 48 | 204 | | | CC or GG |
| NC0009449 | 99.7 | | 49 | 188 | | | AA or GG |
| NC0033372 | 96.8 | | 50 | 237 | | | AA or CC |
| NC0105925 | 96.9 | 319.3 | 51 | 267 | | | AA or GG |
| NC0110365 | 97 | 319.7 | 52 | 425 | | | AA or GG |
| NC0036506 | 100 | 335.7 | 53 | 79 | | | CC or TT |
| NC0016876 | 89.2 | 276.3 | 54 | 89 | | | AA or GG |
| NC0039812 | 92.7 | 285.8 | 55 | 73 | | | |

¹IBM2 Neighbors 2008 Genetic Map. World Wide Web. (mgdb.com)
² Alleles of the single nucleotide polymorphisms that can be associated with an haploid induction phenotype are shown.³

### IV. Methods and Uses for Haploid Mapping

In certain embodiments, the present invention comprises identification and introgression of QTL associated with desirable traits using haploid plants in a plant breeding program. In one aspect, the present invention includes methods and compositions for mapping disease resistance loci in maize.

The present invention provides a method of using haploid plants to identify genotypes associated with phenotypes of interest wherein the haploid plant is assayed with at least one marker and associating the at least one marker with at least one phenotypic trait. The genotype of interest can then be used to make decisions in a plant breeding program. Such decisions include, but are not limited to, selecting among new breeding populations which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germplasm improvement activities based on presence of favorable nucleic acid sequences.

Non-limiting examples of germplasm improvement activities include line development, hybrid development, transgenic event selection, making breeding crosses, testing and advancing a plant through self fertilization, using plants for transformation using plants for candidates for expression constructs, and using plants for mutagenesis.

Non-limiting examples of breeding decisions include progeny selection, parent selection, and recurrent selection for at least one haplotype. In another aspect, breeding decisions relating to development of plants for commercial release comprise advancing plants for testing, advancing plants for purity, purification of sublines during development, inbred development, variety development, and hybrid development. In yet other aspects, breeding decisions and germplasm improvement activities comprise transgenic event selection, making breeding crosses, testing and advancing a plant through self-fertilization, using plants for transformation, using plants for candidates for expression constructs, and using plants for mutagenesis.

In still another embodiment, the present invention acknowledges that preferred haplotypes and QTL identified by the methods presented herein may be advanced as candidate genes for inclusion in expression constructs, i.e., transgenes. Nucleic acids underlying haplotypes or QTL of interest may be expressed in plant cells by operably linking them to a promoter functional in plants. In another aspect, nucleic acids underlying haplotypes or QTL of interest may have their expression modified by doublestranded RNA-mediated gene suppression, also known as RNA interference (“RNAi”),which includes suppression mediated by small interfering RNAs (“siRNA”), trans-acting small interfering RNAs (Ita-siRNA”), or microRNAs (“miRNA”). Examples of RNAi methodology suitable for use in plants are described in detail in U. S. Patent Application Publications 2006/0200878 and 2007/0011775.

Methods are known in the art for assembling and introducing constructs into a cell in such a manner that the nucleic acid molecule for a trait is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd Edition Volumes 1, 2, and 3(2000) J. F. Sambrook, D. W. Russell, and N. Irwin, Cold Spring Harbor Laboratory Press. Methods for making transformation constructs particularly suited to plant transformation include, without limitation, those described in U.S. Pat. Nos. 4,971,908, 4,940,835, 4,769,061 and 4,757,011, all of which are herein incorporated by reference in their entirety. Transformation methods for the introduction of expression units into plants are known in the art and include electroporation as illustrated in U.S. Pat. No. 5,384,253;microprojectile bombardment as illustrated in U.S. Pat. Nos. 5,015,580; 5,550,318; 5,538,880; 6,160,208; 6,399,861; and 6,403,865; protoplast transformation as illustrated in U.S. Pat. No. 5,508,184; and Agrobacterium-mediated transformation as illustrated in U.S. Pat. Nos. 5,635,055; 5,824,877; 5,591,616; 5,981,840; and 6,384,301.

The method of the present invention can be used to identify genotypes associated with phenotypes of interest such as those associated with disease resistance, herbicide tolerance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for consumer appeal.

The method of the present invention facilitates the production of DH plants, which entails induction of haploidization followed by diploidization, which requires a high input of resources. DH plants rarely occur naturally, therefore, artificial means of production are used. First one or one or more lines are crossed with an inducer parent to produce haploid seed. Inducer lines for maize include Stock 6, RWS, KEMS, KMS and ZMS, and indeterminate gametophyte (ig) mutation. Selection of haploid seed can be accomplished by various screening methods based on phenotypic or genotypic characteristics. In one approach, material is screened with visible marker genes, including GFP, GUS, anthocyanin genes such as R-nj, luciferase, YFP, CFP, or CRC, that are only induced in the endosperm cells of haploid cells, allowing for separation of haploid and diploid seed. Other screening approaches include chromosome counting, flow cytometry, and genetic marker evaluation can be utilized to infer copy number.

Resulting haploid seed has a haploid embryo and a normal triploid endosperm. There are several approaches known in the art to achieve chromosome doubling. Haploid cells, haploid embryos, haploid seeds, haploid seedlings, or haploid plants can be treated with a doubling agent. Non-limiting examples of known doubling agents include nitrousoxide gas, anti-microtubule herbicides, anti-microtubule agents, colchicine, pronamide, and mitotic inhibitors.

The present invention includes methods for breeding crop plants such as maize(Zea mays).

It is appreciated by one skilled in the art that haploid plants can be generated from any generation of plant population and that the methods of the present invention can be used with one or more individuals from any generation of plant population. Non-limiting examples of plant populations include F1, F2, BC1, BC2F1, F3:F4, F2:F3, and so on, including subsequent filial generations, as well as experimental populations such as RILs and NILs. It is further anticipated that the degree of segregation within the one or more plant populations of the present invention can vary depending on the nature of the trait and germplasm under evaluation.

For the purpose of haploid QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTL, particularly in the case of haplotypes.

For the purpose of haploid QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTL, particularly in the case of haplotypes. Selection of appropriate mapping populations is important to map construction. The choice of an appropriate mapping population depends on the type of marker systems employed (Tanksley et al., Molecular mapping in plant chromosomes. Chromosome structure and function: Impact of new concepts J. P. Gustafson and R. Appels (eds.).Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted x exotic) and generally yield greatly reduced linkage distances. Wide crosses will usually provide segregating populations with a relatively large array of polymorphisms when compared to progeny in a narrow cross (adapted x adapted).

Maximum genetic information is obtained from a completely classified F2 population using a codominant marker system (Mather, Measurement of Linkage in Heredity: Methuen and Co., (1938)). In the case of dominant markers, progeny tests (e.g. F3,BCF2) are required to identify the heterozygotes, thus making it equivalent to a completely classified F2 population. However, this procedure is often prohibitive because of the cost and time involved in progeny testing. Progeny testing of F2 individuals is often used in map construction where phenotypes do not consistently reflect genotype (e.g. disease resistance) or where trait expression is controlled by a QTL. Segregation data from progeny test populations (e.g. F3 or BCF2) can be used in map construction. Marker-assisted selection can then be applied to cross progeny based on marker-trait map associations (F2′ F3), where linkage groups have not been completely disassociated by recombination events (i.e., maximum disequilibrium).

Further, the present invention contemplates that preferred haploid plants comprising at least one genotype of interest are identified using the methods disclosed in U.S. Patent Application Ser. No. 60/837,864, which is incorporated herein by reference in its entirety, wherein a genotype of interest may correspond to a QTL or haplotype and associated with at least one phenotype of interest. The methods include association of at least one haplotype with at least one phenotype, wherein the association is represented by a numerical value and the numerical value is used in the decision-making of a breeding program. Non-limiting examples of numerical values include haplotype effect estimates, haplotype frequencies, and breeding values. In the present invention, it is particularly useful to identify haploid plants of interest based on at least one genotype, such that only those lines undergo doubling, which saves resources. Resulting doubled haploid plants comprising at least one genotype of interest are then advanced in a breeding program for use in activities related to germplasm improvement.

### V. Marker-Assisted Selection (MAS)

In the present invention, haplotypes are defined on the basis of one or more polymorphic markers within a given haplotype window, with haplotype windows being distributed throughout the crop's genome. In another aspect, de novo and/or historical marker-phenotype association data are leveraged to infer haplotype effect estimates for one or more phenotypes for one or more of the haplotypes for a crop. Haplotype effect estimates enable one skilled in the art to make breeding decisions by comparing haplotype effect estimates for two or more haplotypes. Polymorphic markers, and respective map positions, of the present invention are provided in U.S. Patent Applications 2005/10204780, 2005/10216545, and 2005/10218305 and which are incorporated herein by reference in their entirety.

In yet another aspect, haplotype effect estimates are coupled with haplotype frequency values to calculate a haplotype breeding value of a specific haplotype relative to other haplotypes at the same haplotype window, or across haplotype windows, for one or more phenotypic traits. In other words, the change in population mean by fixing the haplotype is determined. In still another aspect, in the context of evaluating the effect of substituting a specific region in the genome, either by introgression or a transgenic event, haplotype breeding values are used as a basis in comparing haplotypes for substitution effects. Further, in hybrid crops, the breeding value of haplotypes is calculated in the context of at least one haplotype in a tester used to produce a hybrid. Once the value of haplotypes at a given haplotype window are determined and high density fingerprinting information is available on specific varieties or lines, selection can be applied to these genomic regions using at least one marker in the at least one haplotype.

In the present invention, selection can be applied at one or more stages of a breeding program: Among genetically distinct populations, herein defined as “breeding populations,” as a pre-selection method to increase the selection index and drive the frequency of favorable haplotypes among breeding populations, wherein pre-selection is defined as selection among populations based on at least one haplotype for use as parents in breeding crosses, and leveraging of marker-trait association identified in previous breeding crosses. A) Among segregating progeny from a breeding population, to increase the frequency of the favorable haplotypes for the purpose of line or variety development. B) Among segregating progeny from a breeding population, to increase the frequency of the favorable haplotypes prior to QTL mapping within this breeding population. C) F or hybrid crops, among parental lines from different heterotic groups to predict the performance potential of different hybrids.

In the present invention, it is contemplated that methods of determining associations between genotype and phenotype in haploid plants can be performed based on haplotypes, versus markers alone (Fan et aI., 2006 Genetics). A haplotype is a segment of DNA in the genome of an organism that is assumed to be identical by descent for different individuals when the knowledge of identity by state at one or more loci is the same in the different individuals, and that the regional amount of linkage disequilibrium in the vicinity of that segment on the physical or genetic map is high. A haplotype can be tracked through populations and its statistical association with a given trait can be analyzed. By searching the target space for a QTL association across multiple QTL mapping populations that have parental lines with genomic regions that are identical by descent, the effective population size associated with QTL mapping is increased. The increased sample size results in more recombinant progeny which increases the precision of estimating the QTL position.

Thus, a haplotype association study allows one to define the frequency and the type of the ancestral carrier haplotype. An “association study” is a genetic experiment where one tests the level of departure from randomness between the segregation of alleles at one or more marker loci and the value of individual phenotype for one or more traits. Association studies can be done on quantitative or categorical traits, accounting or not for population structure and/or stratification. In the present invention, associations between haplotypes and phenotypes for the determination of “haplotype effect estimates” can be conducted de novo, using mapping populations for the evaluation of one or more phenotypes, or using historical genotype and phenotype data.

A haplotype analysis is important in that it increases the statistical power of an analysis involving individual biallelic markers. In a first stage of a haplotype frequency analysis, the frequency of the possible haplotypes based on various combinations of the identified bi-allelic markers of the invention is determined. The haplotype frequency is then compared for distinct populations and a reference population. In general, any method known in the art to test whether a trait and a genotype show a statistically significant correlation may be used.

Methods for determining the statistical significance of a correlation between a phenotype and a genotype, in this case a haplotype, may be determined by any statistical test known in the art and with any accepted threshold of statistical significance being required. The application of particular methods and thresholds of significance are well within the skill of the ordinary practitioner of the art.

To estimate the frequency of a haplotype, the base reference germplasm has to be defined (collection of elite inbred lines, population of random mating individuals, etc.) and a representative sample (or the entire population) has to be genotyped. For example, in one aspect, haplotype frequency is determined by simple counting if considering a set of inbred individuals.

In another aspect, estimation methods that employ computing techniques like the Expectation Maximization (EM) algorithm are required if individuals genotyped are heterozygous at more than one locus in the segment and linkage phase is unknown (Excoffier et al., 1995 Mol. Biol. Evol. 12:921-927; Li et al. 2002Biostatistics). Preferably, a method based on the EM algorithm (Dempster et aI., 1977 J. R. Stat. Soc. Ser. B 39:1-38) leading to maximum-likelihood estimates of haplotype frequencies under the assumption of Hardy-Weinberg proportions (random mating) is used (Excoffier et al., 1995 Mol. Biol. Evol. 12:921-927). Alternative approaches are known in the art that for association studies: genome-wide association studies, candidate region association studies and candidate gene association studies (Li et al. 2006 BMC Bioinformatics 7:258). The polymorphic markers of the present invention may be incorporated in any map of genetic markers of a plant genome in order to perform genome-wide association studies.

The present invention comprises methods to detect an association between at least one haplotype in a haploid crop plant and a preferred trait, including a trans gene, or a multiple trait index and calculate a haplotype effect estimate based on this association. In one aspect, the calculated haplotype effect estimates are used to make decisions in a breeding program. In another aspect, the calculated haplotype effect estimates are used in conjunction with the frequency of the at least one haplotype to calculate a haplotype breeding value that will be used to make decisions in a breeding program. A multiple trait index (MTI) is a numerical entity that is calculated through the combination of single trait values in a formula. Most often calculated as a linear combination of traits or normalized derivations of traits, it can also be the result of more sophisticated calculations (for example, use of ratios between traits). This MTI is used in genetic analysis as if it were a trait.

One skilled in the art will recognize that haplotypes that are rare in the population in which effects are estimated tend to be less precisely estimated, this difference of confidence may lead to adjustment in the calculation. For example one can ignore the effects of rare haplotypes, by calculating breeding value of better known haplotype after adjusting the frequency of these (by dividing it by the sum of frequency of the better known haplotypes). One could also provide confidence intervals for the breeding value of each haplotypes.

In cases where haplotype windows are coincident with segments in which genes have been identified it is possible to deduce with high probability that gene inferences can be extrapolated to other germplasm having an identical genotype, or haplotype, in that haplotype window. This a priori information provides the basis to select for favorable genes or gene alleles on the basis of haplotype identification within a given population. For example, plant breeding decisions could comprise: A) Selection among haploid breeding populations to determine which populations have the highest frequency of favorable haplotypes, wherein haplotypes are designated as favorable based on coincidence with previous gene mapping and preferred populations undergo doubling; or B) Selection of haploid progeny containing the favorable haplotypes in breeding populations, wherein selection is effectively enabled at the gene level, wherein selection could be done at any stage of breeding and at any generation of a selection and can be followed by doubling; or C) Prediction of progeny performance for specific breeding crosses; or D) Selection of haploid plants for doubling for subsequent use in germplasm improvement activities based on the favorable haplotypes, including line development, hybrid development, selection among transgenic events based on the breeding value of the haplotype that the trans gene was inserted into, making breeding crosses, testing and advancing a plant through self fertilization, using plant or parts thereof for transformation, using plants or parts thereof for candidates for expression constructs, and using plant or parts thereof for mutagenesis.

A preferred haplotype provides a preferred property to a parent plant and to the progeny of the parent when selected by a marker means or phenotypic means. The method of the present invention provides for selection of preferred haplotypes, or haplotypes of interest, and the accumulation of these haplotypes in a breeding population.

In the present invention, haplotypes and associations of haplotypes to one or more phenotypic traits, for example, haploid induction, provide the basis for making breeding decisions and germplasm improvement activities.

Non-limiting examples of breeding decisions include progeny selection, parent selection, and recurrent selection for at least one haplotype. In another aspect, breeding decisions relating to development of plants for commercial release comprise advancing plants for testing, advancing plants for purity, purification of sub lines during development, inbred development, variety development, and hybrid development.

In yet other aspects, breeding decisions and germplasm improvement activities comprise transgenic event selection, making breeding crosses, testing and advancing a plant through self-fertilization, using plants or parts thereof for transformation, using plants or parts thereof for candidates for expression constructs, and using plants or parts thereof for mutagenesis.

In another embodiment, this invention enables indirect selection through selection decisions for at least one phenotype based on at least one numerical value that is correlated, either positively or negatively, with one or more other phenotypic traits. For example, a selection decision for any given haplotype effectively results in selection for multiple phenotypic traits that are associated with the haplotype.

In still another embodiment, the present invention acknowledges that preferred haplotypes identified by the methods presented herein may be advanced as candidate genes for inclusion in expression constructs, i.e., transgenes. Nucleic acids underlying haplotypes of interest may be expressed in plant cells by operably linking them to a promoter functional in plants. In another aspect, nucleic acids underlying haplotypes of interest may have their expression modified by double-stranded RNA-mediated gene suppression, also known as RNA interference (“RNAi”), which includes suppression mediated by small interfering RNAs (“siRNA”), trans-acting small interfering RNAs (“ta_siRNA”), or microRNAs (“miRNA”). Examples of RNAi methodology suitable for use in plants are described in detail in U. S. Patent Application Publications 2006/0200878and 2007/0011775. Methods are known in the art for assembling and introducing constructs into a cell in such a manner that the nucleic acid molecule for a trait is transcribed into a functional mRNA molecule that is translated and expressed as a protein product.

For the practice of the present invention, conventional compositions and methods for preparing and using constructs and host cells are well known to one skilled in the art, see for example, Molecular Cloning: A Laboratory Manual, 3rd Edition Volumes 1, 2, and 3(2000) IF. Sambrook, D. W. Russell, and N. Irwin, Cold Spring Harbor Laboratory Press. Methods for making transformation constructs particularly suited to plant transformation include, without limitation, those described in U.S. Pat. Nos. 4,971,908, 4,940,835, 4,769,061 and 4,757,011, all of which are herein incorporated by reference in their entirety. Transformation methods for the introduction of expression units into plants are known in the art and include electroporation as illustrated in U.S. Pat. No. 5,384,253; microprojectile bombardment as illustrated in U.S. Pat. Nos. 5,015,580; 5,550,318; 5,538,880; 6,160,208; 6,399,861; and 6,403,865; protoplast transformation as illustrated in U.S. Pat. No. 5,508,184; and Agrobacterium-mediated transformation as illustrated in U.S. Pat. Nos. 5,635,055; 5,824,877; 5,591,616; 5,981,840; and 6,384,301.

Another preferred embodiment of the present invention is to build additional value by selecting a composition of haplotypes wherein each haplotype has a haplotype effect estimate that is not negative with respect to yield, or is not positive with respect to maturity, or is null with respect to maturity, or amongst the best 50 percent with respect to a phenotypic trait, transgene, and/or a multiple trait index when compared to any other haplotype at the same chromosome segment in a set of germplasm, or amongst the best 50 percent with respect to a phenotypic trait, transgene, and/or a multiple trait index when compared to any other haplotype across the entire genome in a set of germplasm, or the haplotype being present with a frequency of 75 percent or more in a breeding population or a set of germplasm provides evidence of its high value, or any combination of these.

This invention anticipates a stacking of haplotypes from multiple windows into plants or lines by crossing parent plants or lines containing different haplotype regions. The value of the plant or line comprising in its genome stacked haplotype regions is estimated by a composite breeding value, which depends on a combination of the value of the traits and the value of the haplotype(s) to which the traits are linked. The present invention further anticipates that the composite breeding value of a plant or line is improved by modifying the components of one or each of the haplotypes. Additionally, the present invention anticipates that additional value can be built into the composite breeding value of a plant or line by selection of at least one recipient haplotype with a preferred haplotype effect estimate or, in conjunction with the haplotype frequency, breeding value to which one or any of the other haplotypes are linked, or by selection of plants or lines for stacking haplotypes by breeding.

Another embodiment of this invention is a method for enhancing breeding populations by accumulation of one or more preferred haplotypes in a set of germplasm. Genomic regions defined as haplotype windows include genetic information that contribute to one or more phenotypic traits of the plant. Variations in the genetic information at one or more loci can result in variation of one or more phenotypic traits, wherein the value of the phenotype can be measured. The genetic mapping of the haplotype windows allows for a determination of linkage across haplotypes.

A haplotype of interest has a DNA sequence that is novel in the genome of the progeny plant and can in itself serve as a genetic marker for the haplotype of interest. Notably, this marker can also be used as an identifier for a gene or QTL. For example, in the event of multiple traits or trait effects associated with the haplotype, only one marker would be necessary for selection purposes. Additionally, the haplotype of interest may provide a means to select for plants that have the linked haplotype region. Selection can be performed by screening for tolerance to an applied phytotoxic chemical, such as an herbicide or antibiotic, or to pathogen resistance. Selection may be performed using phenotypic selection means, such as, a morphological phenotype that is easy to observe such as seed color, seed germination characteristic, seedling growth characteristic, leaf appearance, plant architecture, plant height, and flower and fruit morphology.

The present invention also provides for the screening of progeny haploid plants for haplotypes of interest and using haplotype effect estimates as the basis for selection for use in a breeding program to enhance the accumulation of preferred haplotypes. The method includes: a) providing a breeding population comprising at least two haploid plants wherein the genome of the breeding population comprises a plurality of haplotype windows and each of the plurality of haplotype windows comprises at least one haplotype; and b) associating a haplotype effect estimate for one or more traits for two or more haplotypes from one or more of the plurality of haplotype windows, wherein the haplotype effect estimate can then be used to calculate a breeding value that is a function of the estimated effect for any given phenotypic trait and the frequency of each of the at least two haplotypes; and c) ranking one or more of the haplotypes on the basis of a value, wherein the value is a haplotype effect estimate, a haplotype frequency, or a breeding value and wherein the value is the basis for determining whether a haplotype is a preferred haplotype, or haplotype of interest; and d) utilizing the ranking as the basis for decision-making in a breeding program; and e) at least one progeny haploid plant is selected for doubling on the basis of the presence of the respective markers associated with the haplotypes of interest, wherein the progeny haploid plant comprises in its genome at least a portion of the haplotype or haplotypes of interest of the first plant and at least one preferred haplotype of the second plant; and f) using resulting doubled haploid plants in activities related to germplasm improvement wherein the activities are selected from the group consisting of line and variety development, hybrid development, transgenic event selection, making breeding crosses, testing and advancing a plant through self fertilization, using plant or parts thereof for transformation, using plants or parts thereof for candidates for expression constructs, and using plant or parts thereof for mutagenesis.

Using this method, the present invention contemplates that haplotypes of interest are selected from a large population of plants, and the selected haplotypes can have a synergistic breeding value in the germplasm of a crop plant. Additionally, this invention provides for using the selected haplotypes in the described breeding methods to accumulate other beneficial and preferred haplotype regions and to be maintained in a breeding population to enhance the overall germplasm of the crop plant.

### VI. Molecular Markers and Marker-Assisted Selection (MAS)

Selected, non-limiting approaches for breeding the plants of the present invention are set forth below. A breeding program can be enhanced using marker assisted selection (MAS) on the progeny of any cross. It is understood that nucleic acid markers of the present invention can be used in a MAS (breeding) program. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

Genotyping can be further economized by high throughput, non-destructive seed sampling. In one embodiment, plants can be screened for one or more markers, such as genetic markers, using high throughput, non-destructive seed sampling.

In a preferred aspect, haploid seed is sampled in this manner and only seed with at least one marker genotype of interest is advanced for doubling. Apparatus and methods for the high-throughput, non-destructive sampling of seeds have been described which would overcome the obstacles of statistical samples by allowing for individual seed analysis.

For example, U.S. patent application Ser. No. 11/213,430 (filed Aug. 26, 2005); U.S. patent application Ser. No. 11/213,431 (filed Aug. 26, 2005); U.S. patent application Ser. No. 11/213,432 (filed Aug. 26, 2005); U.S. patent application Ser. No. 11/213,434 (filed Aug. 26, 2005); and U.S. patent application Ser. No.11/213,435 (filed Aug. 26, 2005), U.S. patent application Ser. No. 11/680,611 (filed Mar. 2, 2007), which are incorporated herein by reference in their entirety, disclose apparatus and systems for the automated sampling of seeds as well as methods of sampling, testing and bulking seeds.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred aspect, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

Breeding lines can be tested and compared to appropriate standards in environments representative of the commercial target area(s) for two or more generations. The best lines are candidates for new commercial cultivars; those still deficient in traits may be used as parents to produce new populations for further selection.

The development of new elite com hybrids requires the development and selection of elite inbred lines, the crossing of these lines and selection of superior hybrid crosses. The hybrid seed can be produced by manual crosses between selected male fertile parents or by using male sterility systems. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Backcross breeding has been used to transfer genes for a simply inherited, highly heritable trait into a desirable homozygous cultivar or inbred line, which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have most attributes of the recurrent parent (e.g., cultivar) and, in addition, the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Descriptions of other breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of Crop Improvement,” Longman, Inc., NY, 369-399, 1979; Sneep and Hendriksen, “Plant Breeding Perspectives,” Wageningen (ed), Center for Agricultural Publishing and Documentation, 1979; Fehr, In: Soybeans: Improvement, Production and Uses, 2nd Edition, Monograph., 16:249, 1987; Fehr, “Principles of Variety Development,”Theory and Technique, (Vol. 1) and Crop Species Soybean (Vol. 2), Iowa State Univ., Macmillan Pub. Co., NY, 360-376, 1987). An alternative to traditional QTL mapping involves achieving higher resolution by mapping haplotypes, versus individual markers (Fan et al., 2006 Genetics 172:663-686).

This approach tracks blocks of DNA known as haplotypes, as defined by polymorphic markers, which are assumed to be identical by descent in the mapping population. This assumption results in a larger effective sample size, offering greater resolution of QTL. Methods for determining the statistical significance of a correlation between a phenotype and a genotype, in this case a haplotype, may be determined by any statistical test known in the art and with any accepted threshold of statistical significance being required. The application of particular methods and thresholds of significance are well within the skill of the ordinary practitioner of the art. It is further understood, that the present invention provides bacterial, viral, microbial, insect, mammalian and plant cells comprising the nucleic acid molecules of the present invention.

As used herein, a “nucleic acid molecule,” be it a naturally occurring molecule or otherwise may be “substantially purified”, if desired, referring to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

The agents of the present invention may be labeled with reagents that facilitate detection of the agent (e.g. fluorescent labels (Prober et al., 1987 Science 238:336-340; Albarella et al., European Patent 144914), chemical labels (Sheldon et al., U.S. Pat. No. 4,582,789; Albarella et al., U.S. Pat, No. 4,563,417), modified bases (Miyoshi et al., European Patent 119448).

The present invention provides methods to identify and use QTL and haplotype information by screening haploid material that enables a breeder to make informed breeding decisions. The methods and compositions of the present invention enable the determination of at least one genotype of interest from one or more haploid plants. In another aspect, a haploid plant comprising at least one genotype of interest can undergo doubling and be advanced in a breeding program. In yet another aspect, a priori QTL and haplotype information can be leveraged, as disclosed in U.S. Patent Application Ser. No. 60/837,864, which is incorporated herein by reference in its entirety, using markers underlying at least one haplotype window, and the resulting fingerprint is used to identify the haplotypic composition of the haplotype window which is subsequently associated with one or more haplotype effect estimates for one or more phenotypic traits as disclosed therein. This information is valuable in decision-making for a breeder because it enables a selection decision to be based on estimated phenotype without having to phenotype the plant per se. Further, it is preferred to make decisions based on genotype rather than phenotype due the fact phenotype is influenced by multiple biotic and abiotic factors that can confound evaluation of any given trait and performance prediction. As used herein, the invention allows the identification of one or more preferred haploid plants such that only preferred plants undergo the doubling process, thus economizing the DH process.

In another aspect, one or more haplotypes are determined by genotyping one or more haploid plants using markers for one or more haplotype windows. The breeder is able to correspond the haplotypes with their respective haplotype effect estimates for one or more phenotypes of interest and make a decision based on the preferred haplotype. Haploid plants comprising one or more preferred haplotypes are doubled using one or more methods known in the art and then advanced in the breeding program.

In one aspect, advancement decisions in line development breeding are traditionally made based on phenotype, wherein decisions are made between two or more plants showing segregation for one or more phenotypic traits. An advantage of the present invention is the ability to make decisions based on haplotypes wherein a priori information is leveraged, enabling “predictive breeding.” In this aspect, during line development breeding for a crop plant, sub lines are evaluated for segregation at one or more marker loci. Individuals segregating at one or more haplotype windows can be identified unambiguously using genotyping and, for any given haplotype window, individuals comprising the preferred haplotype are selected. In preferred aspects, the selection decision is based on a haplotype effect estimate, a haplotype frequency, or a breeding value.

In another embodiment, at least one preferred nucleic acid of the present invention is stacked with at least one transgene. In another aspect, at least one transgenic event is advanced based on linkage with or insertion in a preferred nucleic acid, as disclosed in published U.S. Patent Application US 2006/0282911, which is incorporated herein by reference in its entirety.

In still another embodiment, the present invention acknowledges that preferred nucleic acids identified by the methods presented herein may be advanced as candidate genes for inclusion in expression constructs, i.e., transgenes. Nucleic acids of interest may be expressed in plant cells by operably linking them to a promoter functional in plants. In another aspect, nucleic acids of interest may have their expression modified by double-stranded RNA-mediated gene suppression, also known as RNA interferences (“RNAi”), which includes suppression mediated by small interfering RNAs (“siRNA”), trans-acting small interfering RNAs (“ta-siRNA”), or microRNAs (“miRNA”). Examples of RNAi methodology suitable for use in plants are described in detail in U.S. patent application publications 2006/0200878 and 2007/0011775.

Methods are known in the art for assembling and introducing constructs into a cell in such a manner that the nucleic acid molecule for a trait is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making transformation constructs particularly suited to plant transformation include, without limitation, those described in U.S. Pat. Nos. 4,971,908, 4,940,835, 4,769,061 and 4,757,011, all of which are herein incorporated by reference in their entirety. Transformation methods for the introduction of expression units into plants are known in the art and include electroporation as illustrated in U.S. Pat. No. 5,384,253; microprojectile bombardment as illustrated in U.S. Pat. Nos. 5,015,580; 5,550,318; 5,538,880; 6,160,208; 6,399,861; and 6,403,865; protoplast transformation as illustrated in U.S. Pat. No. 5,508,184; and Agrobacterium-mediated transformation as illustrated in U.S. Pat. Nos. 5,635,055; 5,824,877; 5,591,616; 5,981,840; and 6,384,301.

### VII. Molecular Assisted Breeding Techniques

Genetic markers that can be used in the practice of the instant invention include, but are not limited to, are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers can form a basis for determining associations with phenotype and can be used to drive genetic gain. The implementation of marker-assisted selection is dependent on the ability to detect underlying genetic differences between individuals.

Certain genetic markers for use in the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multi-allelic, codominant markers often become more informative of the genotype than dominant markers.

In another embodiment, markers that include, but are not limited, to single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with haploid induction loci, regions flanking haploid induction loci, regions linked to haploid induction loci, and/or regions that are unlinked to haploid induction loci can be used in certain embodiments of the instant invention.

In one embodiment, nucleic acid-based analyses for determining the presence or absence of the genetic polymorphism (i.e. for genotyping) can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (Genotypes) that comprise or are linked to a genetic marker that is linked to or correlated with haploid induction loci, regions flanking haploid induction loci, regions linked to haploid induction loci, and/or regions that are unlinked to haploid induction loci can be used in certain embodiments of the instant invention.

Herein, nucleic acid analysis methods include, but are not limited to, PCR-based detection methods (for example, TaqMan assays), microarray methods, mass spectrometry-based methods and/or nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. However, the compositions and methods of the present invention can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleoside triphosphates. If the polymorphism is present on the template, one of the labeled dideoxynucleoside triphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R. F. Service Science 2006 311:1544-1546.

The markers to be used in the methods of the present invention should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs, particularly in the case of genotypes.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention.

### Example 1

### Selection of a Genomic Locus to Increase Haploidization

The present invention provides a method to select for a haploid induction locus to increase induction frequency. The locus was selected for and against using DNA markers on chromosome 1. Bulk groups were created for each population using seed chipping technology on the following five gynogenetic haploid induction populations.

TABLE 2
Five gynogenetic haploid induction populations.

| Generation | Abbreviated Pedigree |
|---|---|
| F3 | HOB:533/FO Beta Version |
| BC1F2 | HOB:007*2/Inducer |
| BC1F2 | HOB:351*2/Inducer |
| BC1F2 | HOB:561*2/Inducer |
| F3 | HOIXXX/Inducer |

Individually selected plants from each population were cross pollinated onto an F1 tester. The haploid induction frequency for each selected plant was determined by visually screening the test cross F1 seed utilizing the R1-nj kernel marker system. The following table documents the number of kernels that were screened for each bulk group and the corresponding haploid induction frequency for each population. Individually selected plants from each population were cross pollinated onto the F1 tester. The haploid induction frequency for each selected plant was determined by visually screening the test cross F1 seed utilizing the R1-nj kernel marker system. The following table documents the number of kernels that were screened for each bulk group and the corresponding haploid induction frequency for each population.

TABLE 3
Kernels screened for each bulk group

| | Haploid | Diploid | Induction | |
|---|---|---|---|---|
| Populations | Kernels | Kernels | Frequency | Locus |
| HOB:533/FO_BETA | 450 | 11218 | 3.9 | Induction Locus |
| HOB:533/FO_BETA | 142 | 22387 | 0.6 | Non-induction |
| | | | | Locus |
| HOB:007*2/Inducer | 151 | 6696 | 2.2 | Induction Locus |
| HOB:007*2/Inducer | 361 | 26418 | 1.3 | Non-induction |
| | | | | Locus |
| HOB:351*2/Inducer | 79 | 816 | 8.8 | Induction Locus |
| HOB:351*2/Inducer | 353 | 7342 | 4.6 | Non-induction |
| | | | | Locus |
| HOB:561*2/Inducer | 349 | 3327 | 9.5 | Induction Locus |
| HOB:561*2/Inducer | 722 | 28901 | 2.4 | Non-induction |
| | | | | Locus |
| XOLU324/Inducer | 363 | 7328 | 4.7 | Induction Locus |

The following table below documents the across populations haploid induction frequency and the across population estimated effect of the desired locus (“Haploid Locus”) using molecular markers for screening.

TABLE 4
Haploid induction frequency across populations.

| | Haploid | | |
|---|---|---|---|
| Populations | Kernels | Diploid Kernels | Induction Frequency |
| Haploid Locus | 1392 | 29385 | 4.5 |
| Non-Haploid Locus | 1788 | 105640 | 1.7 |
| | | Estimated Effect | 272% |

### Example 2

### Pre-Selection of Haploids for Doubling

The utility of haploid plants in genetic mapping of traits of interest is demonstrated in the following example. A haploid mapping population is developed by inducing a family based pedigree, such as an F3 or BC1F2, to produce haploid seeds. The haploid seeds are planted in ear rows which represent the parents from the F3 or BCF2 population and remnant seed is stored for doubling after phenotyping is completed. For mapping, SNP markers are used to screen the putative haploid population. Composite interval mapping is conducted to examine significant associations between a trait of interest and the SNP markers. Such traits can include but are not limited to, disease resistance, herbicide tolerance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for consumer appeal. Remnant seed can be doubled through methods known in the art. Genotypic and phenotypic data can be used in selection of which remnant seed families to double. Doubled plants can be utilized for further breeding, commercial breeding or for additional fine-mapping purposes.

### Example 3

### Genetic Mapping of the Haploid Induction Locus

The haploid induction locus was fine mapped using a panel of molecular markers located genomic region. Bulk populations were developed from an inbred x inducer crosses and each bulk was characterized based on the genotype at each of the listed molecular markers in Table below. Molecular markers designated as “+” were haploid, Molecular markers designated as “−” were diploid. The recombinants in a desirable bulk (for example, Bulk 3 in this experiment), were further analyzed. The selected recombinants are further selfed for a number of generations and backcrossed to increase the resolution for sequencing purposes. These recombinants can also be used for breeding with reduced linkage drag.

TABLE 5
Recombinant bulks for haploid induction locus.

| | Chromosome | | | | |
|---|---|---|---|---|---|
| | 1 | 1 | 1 | 1 | 1 |
| | Position | | | | |
| | 89.2 | 92.7 | 97 | 96.9 | 100 |
| | Marker | | | | |
| | NC0016876 | NC0039812 | NC0110365 | NC0105925 | NC0043554 |
| Bulk Group 1 | +/+ | +/+ | +/+ | +/+ | +/+ |
| Bulk Group 2 | − | − | − | − | − |
| Bulk Group 4 | + | − | − | − | − |
| Bulk Group 6 | + | + | − | − | − |
| Bulk Group 8 | + | + | + | − | − |
| Bulk Group 10 | + | + | + | + | − |
| Bulk Group 3 | + | + | + | + | + |
| Bulk Group 11 | − | + | + | + | + |
| Bulk Group 9 | − | − | + | + | + |
| Bulk Group 7 | − | − | − | + | + |
| Bulk Group 5 | − | − | − | − | + |

### Example 4

### Exemplary Marker Assays for Detecting Ploidy

In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means. Exemplary primers and probes for amplifying and detecting genomic regions associated with a stem canker resistance phenotype are given in Table 9.

TABLE 6
Exemplary Assays for Detecting Polymorphisms

| | | | SEQ ID | SEQ ID | | |
|---|---|---|---|---|---|---|
| Marker or | Marker | SNP | Forward | Reverse | SEQ ID | SEQ ID |
| Locus Name | SEQ ID | Position | Primer | Primer | Probe 1 | Probe 2 |
| NC0016876 | 54 | 293 | TCCGAGC | GCTGGAC | TGAGGCA | AGGCAAG |
| | | | TGGTCAC | AGGTGGA | AACACTC | CACTCC |
| | | | GCA | TGATCTG | | |
| NC0039812 | 55 | 73 | GTGTCTT | CATATGA | CGCATAA | ACGCATA |
| | | | TTGGATA | GCACGGA | CAGTAAA | ACTGTAA |
| | | | GACTGAT | GCACAGA | CA | ACA |
| | | | AGTGATA | A | | |
| | | | GG | | | |
| NC0105925 | 51 | 267 | CCCATTT | GGCACGG | ACAGCTT | CAGCTCC |
| | | | CTGACGT | GATCTGA | CACGCGG | ACGCGGT |
| | | | GAATTTC | AGAGAA | T | |
| | | | TG | | | |

### Example 5

### Use of Identified Haploid Seed for Pre-Selection in a High Oil Breeding Program

The methods of the present invention can be used in a high oil corn breeding program. Haploid kernels with at least one preferred marker, such as oil content, can be selected according to the present invention. Pre-selection breeding methods are utilized to preselect and prescreen lines for oil and agronomic traits such as yield, using markers selected from the group consisting of genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

Populations are identified for submission to the doubled haploid (DH) process. QTL and/or genomic regions of interest are identified in one or more parents in the population for targets of selection that are associated with improved agronomic trait such as yield, moisture, and test weight. In other aspects, QTL are identified that are associated with improved oil composition and/or increased oil composition. In one aspect, two or more QTL may be selected.

Populations are identified for submission to the doubled haploid (DH) process. QTL and/or genomic regions of interest are identified in one or more parents in the population for targets of selection that are associated with improved agronomic traits such as yield, moisture, and test weight. In other aspects, QTL are identified that are associated with improved oil composition and/or increased oil composition. In one aspect, two or more QTL may be selected. The population undergoing haploid induction can be characterized for oil content using methods known in the art, non-limiting examples of which include NIT, NIR, NMR, and MRI, wherein seed is measured in a bulk and/or on a single seed basis. Methods to measure oil content in single seeds have been described (Kotyk, 1., et al., Journal of American Oil Chemists' Society 82: 855-862 (2005). In one aspect, single kernel analysis (SKA) is conducted via magnetic resonance or other methods. In another aspect, oil content is measured using analytics methods known in the art per ear and the selected ears are bulked before undergoing SKA. The resulting data is used to select single kernels that fall within an oil range acceptable by the breeder to meet the product concept.

The seed samples are genotyped using the markers corresponding to the one or more QTL of interest. Seeds are selected based upon their genotypes for these QTL.

Seed may be selected based on preferred QTL alleles or, for the purpose of additional mapping, both ends of the distribution are selected. That is, seed is selected based on preferred and less preferred alleles for at least one QTL and/or preferred and less preferred phenotypic performance for at least one phenotype and/or preferred and less preferred predicted phenotypic performance for at least one phenotype. Haploid kernels can also be selected and processed by methods known in the art.

Seed may be selected based on preferred QTL alleles or, for the purpose of additional mapping, both ends of the distribution are selected. That is, seed is selected based on preferred and less preferred alleles for at least one QTL and/or preferred and less preferred phenotypic performance for at least one phenotype and/or preferred and less preferred predicted phenotypic performance for at least one phenotype. Haploid kernels can also be selected and processed by methods known in the art. such as NMR or MRI to characterize oil content. Kernels with preferred oil content are selected. As illustrated above, for research purposes, kernels may be selected with low, high, or average oil content in order to identify the genetic basis for oil content. In one aspect, relative oil content in germ and endosperm is characterized by taking an NMR measurement on whole kernel, wherein subsequent NMR measurements are taken on dissected germ and endosperm. In another aspect, kernels are imaged using MR1 to identify the relative oil content in germ and endosperm tissue.

### Example 6

### Ploidy Determination in a Breeding Program

In a double haploid breeding program, the recovery of haploid kernels is the result of initiating a cross to an inducer line. The inducer line has unique genomic regions that are associated with the mechanism of induction. The use of SNP markers on chromosome 1 has enabled one skilled in the art to determine ploidy level of the F1 plants resulting from a cross to inducer lines, distinguishing haploid plants from non-haploid plants.

Current methods for distinguishing haploid kernels from diploid kernels in a double haploid breeding program are based on the presence or absence of the visible anthocyanin marker in the embryo. This method, however, results in error caused by misclassification or variable anthocyanin marker expression. In an effort to compliment this screening method the use of DNA markers can accurately determine ploidy level minimizing misclassification rate. Another embodiment of this invention will determine accurate rates of induction critical to a double haploid breeding program.

Current methods for distinguishing haploid kernels from diploid kernels in a double haploid breeding program are based on the presence or absence of the visible anthocyanin marker in the embryo. This method, however, results in error caused by misclassification or variable anthocyanin marker expression. In an effort to compliment this screening method the use of DNA markers can accurately determine ploidy level minimizing misclassification rate. Another embodiment of this invention will determine accurate rates of induction critical to a double haploid breeding program. The use of DNA markers may be used to improve the efficiency of the doubled haploid program through selection of desired genotypes at the haploid stage and identification of ploidy level to eliminate non-haploid seeds from being processed and advancing to the field. Both applications again result in the reduction of field resources per population and the capability to evaluate a large number of populations within a given field unit.

Selected kernels will be grown to a desirable plant stage and DNA markers can be utilized to accurately determine ploidy levels while minimizing misclassification of haploid to non-haploid seeds. Extracted DNA from plant tissue or seed embryos is screened for the presence or absence of a suitable genetic marker selected from on chromosome 1.

We claim:

1. A method of identifying a maize plant that comprises a genotype associated with an increased haploid induction phenotype, comprising:
i) detecting in a maize plant an allele in at least one haploid induction locus associated with an increased haploid induction phenotype wherein the haploid induction locus is on chromosome 1 in a genomic region flanked by or including:
a) loci NC0016876 (SEQ ID NO: 54) and NC0039812 (SEQ ID NO: 55);
b) loci NZMAY008358670 (SEQ ID NO:18) and loci NC0039812 (SEQ ID NO:55); or
c) loci NC0016876 (SEQ ID NO:54) and loci NZMAY008358232 (SEQ ID NO:26); and
ii) denoting that said maize plant comprises a genotype associated with an increased haploid induction phenotype.

2. The method of claim 1, wherein said method further comprises the step of selecting said denoted maize plant from a population of maize plants.

3. The method of claim 2, wherein said selected maize plant exhibits increased haploid induction when crossed to a tester.

4. The method of any one of claim 1, wherein said genotype associated with an increased haploid induction phenotype comprises at least one polymorphic allele of a marker selected from the group consisting of loci NC0016876 (SEQ ID NO: 54) and loci NC0039812 (SEQ ID NO: 55) .

5. The method of any one of claim 1, wherein said genotype associated with an increased haploid induction phenotype comprises at least one polymorphic allele of at least one marker in chromosome 1 selected from the group consisting of SEQ ID NO: 3, SEQ ID NO: 21, and SEQ ID NO: 32 that is flanked by loci NC0016876 (SEQ ID NO: 54) and NC0039812 (SEQ ID NO: 55) in chromosome 1.

6. The method of claim 5, wherein said genotype associated with an increased haploid induction phenotype comprises a haplotype of at least two markers in chromosome 1 selected from the group consisting of SEQ ID NO: 3, SEQ ID NO: 21, and SEQ ID NO: 32 that is associated with an increased haploid induction phenotype.

7. The method of claim 5, wherein said genotype associated with an increased haploid induction phenotype comprises a haplotype of at least two markers in said chromosome 1 selected from the group consisting of SEQ ID NO: 54, SEQ ID NO: 55, SEQ ID NO: 51, SEQ ID NO: 52, and SEQ ID NO: 53 that is associated with an increased haploid induction phenotype.

8. The method of claim 5, wherein the preferred haplotype is SEQ ID NO: 51 and SEQ ID NO: 52.

9. A method for obtaining a maize plant comprising in its genome at least one haploid induction locus, compromising the steps of:
i. genotyping a plurality of maize plants with respect to at least one haploid induction locus on chromosome 1 in a genomic region flanked by or including:
(a) loci NC0016876 (SEQ ID NO: 54) and loci NC0039812 (SEQ ID NO: 55);
(b) loci NZMAY008358670 (SEQ ID NO:18) and loci NC0039812 (SEQ ID NO: 55); or
(c) loci NC0016876 (SEQ ID NO: 54) and loci NZMAY008358232 (SEQ ID NO: 26); and
ii. selecting a maize plant comprising in its genome at least one haploid induction locus comprising a genotype associated with a increased haploid induction phenotype.

10. The method of claim 9, wherein said selected maize plant exhibits increased haploid induction when crossed to a tester.

11. The method of claim 9, further comprising assaying said selected maize plant of step (ii.) for increased haploid induction.

12. The method of claim 9, further comprising the step of assaying for the presence of at least one additional marker, wherein said additional marker is either linked or unlinked to a genomic region of chromosome 1 and flanked by any one of the loci sets of (a)-(c).

13. The method of claim 9, wherein said haploid induction locus is genotyped for at least one polymorphic allele of a marker selected from the group consisting of SEQ ID NO: 54, SEQ ID NO: 55, SEQ ID NO: 51, SEQ ID NO: 52, and SEQ ID NO: 53.

14. A method of breeding for increased haploid induction in a maize plant, comprising the steps of:
determining the presence of at least one haploid induction loci within the chromosomal region selection between molecular markers SEQ ID NO: 54 to SEQ ID NO: 55 in said maize plant;
selfing or crossing the maize plant in which the region is present; and
determining the presence of a chromosomal region identified in step (a) in the progeny of the selfing or crossing step using one or more molecular markers.

15. A maize plant of claim 14, having a genome comprising of at least one chromosomal region selected from loci NC0016876 (SEQ ID NO: 54) and loci NC0039812 (SEQ ID NO: 55) and demonstrating increased haploid induction.


SEQUENCE LISTING (55 sequences)

SEQ ID NO: 1 - DNA, 201 bp, Zea mays
catatttatatacttattatatttactaggtcagtacccgtgcgttgcaacgggaacatataataccatgataacttatatacaaaatgtgtcttatattgttactaggtgagtgcccgtgcgttgcaacggagacacataatacataagttaccgtgatattatatgtccccgttgcaacgcatggacactcacctatat

SEQ ID NO: 2 - DNA, 201 bp, Zea mays
accgtagagttagcactgattcggcctctaatgccatgtgtcttgctgttgtcatcttctgtatttacgatatgaacaggagtgttcgaattccttcctaccaccacctctctgttaccactgtgaatgataggtgatcactgataaatctacatcaggcttggtaaaagtttcatcgtattttttttcagttgaaaagcg

SEQ ID NO: 3 - DNA, 201 bp, Zea mays
agaaggactttgaatgggtccaaacacatcaacattagcagaaactgcaaccgtagagttagcactgattcggcctctaatgccatgtgtcttgctgttgtcatcttctgtatttacgatatgaacaggagtgttcgaattccttcctaccaccacctctctgttaccactgtgaatgataggtgatcactgataaatcta

SEQ ID NO: 4 - DNA, 201 bp, Zea mays
gagtcaggattgtcttggcctgtaatatcatgggtattgatgtctgcatcttctatattcatgatatgaacagaaggactttgaatgggtccaaacacatcaacattagcagaaactgcaaccgtagagttagcactgattcggcctctaatgccatgtgtcttgctgttgtcatcttctgtatttacgatatgaacagga

SEQ ID NO: 5 - DNA, 201 bp, Zea mays
accaacatatttttctttaacttcctgcagatttgggctatttgtggttcttacagtgtctctcttttcaccagaagattgaatgggagcgagtatatcaaccttggcagaaattggcaacaaactagagtcaggattgtcttggcctgtaatatcatgggtattgatgtctgcatcttctatattcatgatatgaacaga

SEQ ID NO: 6 - DNA, 201 bp, Zea mays
tgatcatggctggcagaatattttcgagaaacatgatctgtatggccttttagaacgtccagggtcccatttaaggtatctacatgatttctaaaaacaggcattttgcttgtgctttgaggacctagaacaccacttgcaaggtcctgtgtccttgcaccaacatatttttctttaacttcctgcagatttgggctattt

SEQ ID NO: 7 - DNA, 201 bp, Zea mays
atcgagctcgtccacagactgatttcctctcaaaggactggtcaataagacttcagcaacatcagtctgatcatggctggcagaatattttcgagaaacatgatctgtatggccttttagaacgtccagggtcccatttaaggtatctacatgatttctaaaaacaggcattttgcttgtgctttgaggacctagaacacc

SEQ ID NO: 8 - DNA, 201 bp, Zea mays
ttcagctgcacatgatgtgctgtagccatttttagaggctgggtcagcatgctcaccatgaatcactttggcaggccaggattcaggaacaactttcttagcactgttgataactgaccctttggcaggccaggattcaggaacaactttcttagcactgttgataactgacccttcagcctcgatatcagcgctccggtg

SEQ ID NO: 9 - DNA, 201 bp, Zea mays
ggcctttgatatggtttttggagaaagtgtttcacagtcactaacacttccatgactggaaagctttttaggccagtcttcaacattgtctgtgggaagcaagtcatcagacaatgcaccagcagccactgtctcagctttgtccattactgtatcattgctcatgttggtgtcagatttccatgacctcttcgtggctct

SEQ ID NO: 10 - DNA, 201 bp, Zea mays
gtatgatccttgcttgtctatcgtgcaagaatcttggcaaaacagccccgacacctctcttaccacatccttgttggacttctcggagtctggatcagcaattactttgctgatattgcatgaaatcccttcggttctgtttgatgaaaaagcttttgtttcagacttaacggcactaccaattttgccaaacttgtcgcc

SEQ ID NO: 11 - DNA, 201 bp, Zea mays
cgccccttcagccgtctaagtatcgagcgataaccccttctctgctcactatttccacttaagataaacaatgcaggtttgctcgtgatcaaggtgcaaggttcacttctatttcccctttggtgcttactgagcacacgcgtgtttttcttgatcgattttgcatcagatttggaacgcagactgccagttctgtttggt

SEQ ID NO: 12 - DNA, 201 bp, Zea mays
actcgccccttcagccgtctaagtatcgagcgataaccccttctctgctcactatttccacttaagataaacaatgcaggtttgctcgtgatcaaggtgcaaggttcacttctatttcccctttggtgcttactgagcacacgcgtgtttttcttgatcgattttgcatcagatttggaacgcagactgccagttctgttt

SEQ ID NO: 13 - DNA, 201 bp, Zea mays
gtatgaccagtgatgtgaatccctacaaactcgccccttcagccgtctaagtatcgagcgataaccccttctctgctcactatttccacttaagataaacaatgcaggtttgctcgtgatcaaggtgcaaggttcacttctatttcccctttggtgcttactgagcacacgcgtgtttttcttgatcgattttgcatcaga

SEQ ID NO: 14 - DNA, 201 bp, Zea mays
acttctcggttctcctcagagggtctggagcgatgaaatgcgttgcttggtatgaccagtgatgtgaatccctacaaactcgccccttcagccgtctaagtatcgagcgataaccccttctctgctcactatttccacttaagataaacaatgcaggtttgctcgtgatcaaggtgcaaggttcacttctatttccccttt

SEQ ID NO: 15 - DNA, 201 bp, Zea mays
ctcccactgataaatggtcatataatcaatgatactaagtactctaagatgtaaagtttgtaaactcttggtagcctaaatcatagtcctaacctgccagctgcagccgctgcaaagaacttctcggttctcctcagagggtctggagcgatgaaatgcgttgcttggtatgaccagtgatgtgaatccctacaaactcgc

SEQ ID NO: 16 - DNA, 201 bp, Zea mays
tatgttctatcagatatcagctttcagttgctcacgctggacttacatcttatcttgttgtgagctacaattaaatgttcaatctcacatacgttcttatccatggatacatttggtcttgaagcagctttgtagtctccttcaagctgcggcaggtgtcgttcgaacttgataaccatgttgtgtagtataatgacacaa

SEQ ID NO: 17 - DNA, 201 bp, Zea mays
gtattccgtgactccatagttgtacatgaactccagaacggttgccttgtccgcgatagtgtaacccaacatggcaatgatatactgcataagggtgaaactgtttagtctactaaactgagtacactacatctatggggagattgcatgccattactctcaacacttagaatacaccaacattttagttcatgtagacca

SEQ ID NO: 18 - DNA, 201 bp, Zea mays
cgcggacaaggacctgaacgcgcgcatgtccttccacacggtggacgtagccaacctgacagatgacctcggcaagtacgacgtcgtcttccttgccgcgctcgtcggcatggcggccgaggacaaggccaaggtggtcgcgcaccttggcaggcacatggcggacggagcggctctcgtcgtgcggagcgcgcacggggc

SEQ ID NO: 19 - DNA, 201 bp, Zea mays
taagattataatatgctcaaattatataatctatctatccctagtttccaagacatgatatgcttcatcaggatagaagggggtcttccgaagctcgtacagttaacagtgtggttctcaatccaagtacatatatcagggtttgaggggctgacctaagatactgaagctgtgcaaagaaaccagaaaatttgtttacat

SEQ ID NO: 20 - DNA, 201 bp, Zea mays
gagaaataaatctggacagacaaatcagcggcaacaacgaaaaaaatctggacaataaaaaaacaaatcagtgacaaagacaaaaaatattgaacactcacagtagggctcgaacccacaaccttaaggttaaaaaccttacgctctaccaactgagctagacaagctttgtggttacacattcaagctcagcgtcataga

SEQ ID NO: 21 - DNA, 201 bp, Zea mays
cttgtggttagtgtatattacttttatcattctataattagtttatatattttaaattcgtttgatctcactaaactctagttttactcatagctgtgcgtctgcgcttactacaagtctacagcctgcaggtagagcgatgtgaccatatattggttcgcaagctgcatgggccaagatttgtataatgacgatttcaaa

SEQ ID NO: 22 - DNA, 201 bp, Zea mays
tcttttgcttgtggttagtgtatattacttttatcattctataattagtttatatattttaaattcgtttgatctcactaaactctagttttactcatagctgtgcgtctgcgcttactacaagtctacagcctgcaggtagagcgatgtgaccatatattggttcgcaagctgcatgggccaagatttgtataatgacga

SEQ ID NO: 23 - DNA, 201 bp, Zea mays
cggacccaaaacaccgacatagtccgtattcttgaaaatgtcatctcagttctcgaaacacgcttctttttttaaaaaaatcttttgcttgtggttagtgtatattacttttatcattctataattagtttatatattttaaattcgtttgatctcactaaactctagttttactcatagctgtgcgtctgcgcttactac

SEQ ID NO: 24 - DNA, 201 bp, Zea mays
tcctctctcatctaccgagatgatactatcctagagcattgcactgcttttagcttcgagtcgtcttaggtttgggtattttttttcaagacctatctaagaggtaacggttcaattagattagaaaaaaaaatattaaagttttttttctaaaagtgacgatagtggaaaatagagaaactagtgctttatgtgctttag

SEQ ID NO: 25 - DNA, 201 bp, Zea mays
ggaaggccagttcagttcttatcatgtcgagtgctttatgtcttgcacttcactgtgtatgtcttctgacaagtatttttgacagattttgacaaggtcattagtcctgtatccgttgcacatttctcacagtacaagttagttgatttgctactcaagaagcttgaaggtattggtttccagacatgctttcccagtgag

SEQ ID NO: 26 - DNA, 201 bp, Zea mays
gaggttgtgacatatttaattgaactttgtagaatatatttatggcataatatatgtattattagaatgtgaattaatatgtgttattgaagtatgacggtagcgtgatcggggctgcagcccgacaacccacctcctagatccgcccctatccatgatggtcatggttattgagtctattaaaagaaaatgagatggtag

SEQ ID NO: 27 - DNA, 201 bp, Zea mays
atggtttttagatatcgtggtttacttgaagataccatacacatgtgtattgaggaacaagtgaccatgttattggaaacagtgggacacaatatggagacaagacaaagcacccttcatgaattaaagatgtcaataattcatggatctctagcaaatgccttctgaatgtgaatcaaattttaggccgctcttgtgcag

SEQ ID NO: 28 - DNA, 201 bp, Zea mays
acttgaagataccatacacatgtgtattgaggaacaagtgaccatgttattggaaacagtgggacacaatatggagacaagacaaagcacccttcatgaattaaagatgtcaataattcatggatctctagcaaatgccttctgaatgtgaatcaaattttaggccgctcttgtgcagccctctaacatttctacgcaagt

SEQ ID NO: 29 - DNA, 201 bp, Zea mays
ataccatacacatgtgtattgaggaacaagtgaccatgttattggaaacagtgggacacaatatggagacaagacaaagcacccttcatgaattaaagatgtcaataattcatggatctctagcaaatgccttctgaatgtgaatcaaattttaggccgctcttgtgcagccctctaacatttctacgcaagtaatttata

SEQ ID NO: 30 - DNA, 201 bp, Zea mays
aataacataagaaaagataaagatcatatctacataaaaaaagataagcatcaaatttcatacatatttattttaaaatattaaatttgttcaaacaattaagatatccacttttatctctagtcacaagccaccaggttcagtttcagttttaaggagccaccagatgcaacatctacaagaaatattaccaaatcgatt

SEQ ID NO: 31 - DNA, 201 bp, Zea mays
aatgggaaatgtcaatatcaattaagtaatagagcagaataatcagagcatactcctctaacaacttccataagacagtcctccaagacaatctgaaggggttcgttcaaattctgatcttcaacattgcctgattccacggcctcaagtgttttccttaagtgcttgcacaagtcacctgccactacaagttcaacagca

SEQ ID NO: 32 - DNA, 201 bp, Zea mays
cgcgaactcgtcattcatcggagagcttgtcgagcagcatccgcatgacctggacctgcgcctgcaagtcctcgatgtaggtcgccgtgtcctggaacagctcgccgagcccgcatggctcacggcacgctggcaccagcctccgaagctccctcacctgcttcagcagtctagggtcctccaccatgtccctctcgccga

SEQ ID NO: 33 - DNA, 201 bp, Zea mays
tataagcgcaggcgcgcgcgcgcgaactcgtcattcatcggagagcttgtcgagcagcatccgcatgacctggacctgcgcctgcaagtcctcgatgtaggtcgccgtgtcctggaacagctcgccgagcccgcatggctcacggcacgctggcaccagcctccgaagctccctcacctgcttcagcagtctagggtcctc

SEQ ID NO: 34 - DNA, 201 bp, Zea mays
tgtcgacggacaaatcctaaatcatgcatggacacatgatcgaagagcagacttcatctcgatcgcgtataagcgcaggcgcgcgcgcgcgaactcgtcattcatcggagagcttgtcgagcagcatccgcatgacctggacctgcgcctgcaagtcctcgatgtaggtcgccgtgtcctggaacagctcgccgagcccgc

SEQ ID NO: 35 - DNA, 201 bp, Zea mays
cgttcgttctcccagtcccagcagtcgagcaacagcttgccaccgtatttcctcaccaacctcctgcagccgctgccgtcgcagcactcactagctcaggctctgccgagctacaacaacttggggttcggggagcccagcttgtactggccctgcccctgcggtgacccgggggagcagaaggtgcagcttggcagcaag

SEQ ID NO: 36 - DNA, 201 bp, Zea mays
gaagactgtggattgctatagtcctggtcaagggttgatgcctgctagtttcaaagttagatctgtacctttagatggcaatagcgaagcatttgaggaggttcttgacccagattttggagagtcagctattggacgtgtagcacctgttgactctggtgagtatttctgtcgaatcatattatagtcatgaagaccaga

SEQ ID NO: 37 - DNA, 201 bp, Zea mays
ccccttccgcgccgccaccctatcgccctctgctccgccgctagagtcggcccggctcgaggatgagcccccgagcagtccgccgccgcaggatgaggcggccgcgtccgtggcggcgcacgagcaggcgacgctggcctgccaggagctggaattggagggcctcaaggcgggggtggaggcggtgaagagcagggagga

SEQ ID NO: 38 - DNA, 201 bp, Zea mays
cgtacctgaatgaaatgcattatgcacatgaatgcatatcgacggaaatattataaatctaaatagtgttacgcgatagcggaacacctagcggcgaggcattgcgactacgcgtaataattacgcttctctaaattaaacgaacctaacaaaaacatcacaaacaacaaatcatatactttaaacataattagcctaatt

SEQ ID NO: 39 - DNA, 201 bp, Zea mays
ctgcggaatattcactcgctcgccggttcacagctaacgcggcaacgatgtggccgagaattgaaaaacccagagctctccctttaaaataccatcacagtaggagcttcgcttacttctctgacctttgaaaacgagtgcggagctgggcgacagggaggcctatctgtctgtccccacatgccagcgtgaagagttcaa

SEQ ID NO: 40 - DNA, 201 bp, Zea mays
gcaacgatgtggccgagaattgaaaaacccagagctctccctttaaaataccatcacagtaggagcttcgcttacttctctgacctttgaaaacgagtgcggagctgggcgacagggaggcctatctgtctgtccccacatgccagcgtgaagagttcaacccggcgctacgacacggacaggggagcagatatcctctca

SEQ ID NO: 41 - DNA, 201 bp, Zea mays
taattaatgcaataggaataagagttgatggttttgaaaaaagatatttctacctacctgtgaactagtgagtccacaagaactatgtacatttgattgatcttgtaccacaggggggctgaaacatccactatcaggaaaattaggtgattgtggtgcagttctaactgtgtcactaatgctggaccctgagcagttaga

SEQ ID NO: 42 - DNA, 201 bp, Zea mays
aaaaaacgaaaatgttcctcacctcagaactgcatgtatcaagagatgtagcagtagactgattatgattggtaaccgaagcatcatccttagagctccggaaccatgttagctcatcatttgattcagatgaaaacaaatcatgattaagagcttgtttatcgatgcttgtagcagaaacataagaaagcccagtgagtg

SEQ ID NO: 43 - DNA, 201 bp, Zea mays
cggaaccatgttagctcatcatttgattcagatgaaaacaaatcatgattaagagcttgtttatcgatgcttgtagcagaaacataagaaagcccagtgagtgcaggctgcttttgttctggaggtgtatcaaatgtggcccacccagcattgtccattgaatgagctgttggtatcaccttatcagcagatggagtttca

SEQ ID NO: 44 - DNA, 201 bp, Zea mays
agcattgtccattgaatgagctgttggtatcaccttatcagcagatggagtttcagtaagcgtatttgaaaatagatctacagattgatctatggtagtgacatggtgttcctgagaagctttggaatcaaagctagcttgatttggagcactaatagcatcagaaaaggctatgaaacttggagcagcggaatttgagga

SEQ ID NO: 45 - DNA, 201 bp, Zea mays
ctgagaagctttggaatcaaagctagcttgatttggagcactaatagcatcagaaaaggctatgaaacttggagcagcggaatttgaggatttttcagttcggtgaacattatcattctcaaaaatcaaatcagataagtttgatttgcctgatttaagagagatagaatctaggtctcctgccgatacagctctctgcag

SEQ ID NO: 46 - DNA, 201 bp, Zea mays
ctgagagctattttcgttcattgttcctttaggctcttgcaatctagaagtcattaatgatagatctcccttgacagtgtactgggtactaccactggaggcatcgaaaatactccaaggctggagaggaagagaaagtaaagaattcaaacagaagatatgaagcataagactaaggccttgtttggcatggtttctctt

SEQ ID NO: 47 - DNA, 584 bp, Zea mays
  unsure (1)..(584): unsure at all n locations
tagcagatccaatcagcagcgatgagtgttcgtttgttattttgattgcgagaactgaaatatgcagcaaggaacgaaaccagacctaggaggacatattaactcatttttaatgatctacacgcatgcgaggatagatatctacacctaacaagttgatacatatttacagtccacagagcctgaactcctaaagcacaaagctaatatagtgtttctgtctccactgagatgcaagttacatgctcgagaatcctgctttagnngnaganccnaatctcacgagacgtttattttagtggtgctctcagcccagattcgcacaggacacaacaggttacagcaaactagtgtgttaattatatcttccttccgtcacccgctattaaaaaaaataatccgactttatatattccgtctcctataattaaaactaatcctctcctgatagcgcatactcctatcaacttttcatccttaccaaaccccttaacccccatttgatccgtatgataccctatcatttaccgataccccctcctccctattcccgtgtatattccttaatcattcgcctattcgct

SEQ ID NO: 48 - DNA, 653 bp, Zea mays
  unsure (1)..(653): unsure at all n locations
aggcatctgatatatagttccgttcgattctgaggccaaagaccttcctatatatagttagttacctttcatgtgttccatggcgtggcagatctcatctttcgccggcgtcttgagctgccgatcggtgaccagctcgangccgagcatgaaacccgttcccctcacgtcgccgatgactgcgtgaacgaacacaggcaaaacaanaanaanaannnnatcaggatcaggcgaggttacgttcgttctggtcagaaagaagaccgaatgttttctgaataaaatccaccagcagctgggccagcagacactcactctcgtgcttctcctggagaccgcggaggcggtccttgaggtaggagccgacgacgaaggcgttctcctggagcctctccttctcgagcacctcgaggacggcgagnccgccggcggtgcacagcgggttgccgccgaacgtgttgaagtagcagcgccgggtcagcacctgcgcgacctccggcgtcgtcaccacggcgcccagcgggatgccgttgccgatgccctgaagaatccaggtgtnnnnccnngttcagagcgnngtgtcgtcgtcgtcgatggcacagaaaaacaannnttggaatggcgcgtacgtacctttgccatgntcactannt

SEQ ID NO: 49 - DNA, 500 bp, Zea mays
aatccgcaactgcaattttctctgaatctgcaatataggaatatatatatatatagtgttaggataaagcaagcttcattggcattaatattaatgttgggcaaaatcacttagggcccgttcgtttgtgtccaggaatggacacaggattcgttccagcttatcaaaacttatataaattagagaaacaatccgactaggaatcgttacaggcctccaatccgtgaaaaccaaacagggccttgtggaatcaacactaactgaatttagaattagtacctcaagctgttcatgaagccgtttctggacttccatctgaaggcgcagtgcttcagtaagatccatgctcctatacaagaatgcccaatatcatggaagagtattaaccaagaagtaacagacaaagatgatcgtaaaatgcactttaacatataggaaaaccttcaaattagattttatgtttcaaaagaaacaacaaaagtgaatgcaatggctcaatg

SEQ ID NO: 50 - DNA, 327 bp, Zea mays
  unsure (1)..(327): unsure at all n locations
atcagtcagcttcatctcatagtagggagatttgtcctgttgctagtcctcccaatagcagcaatgcttcagttgccaaacaacggatgagatggaccccagaactccatgaatgttttgtagatgctgtaaatcagcttggcggtagcgaaagtatgtctnccctattggcatttcccctcaaatagtttaagttatgaaccctacatcacatcatctaatgcatgtgtttatttatcttttcttattcctattcagaagctactcctaagggtgtgctaaagcttatgaaagttgatggtttgactatatatcatgtcaaaagcc

SEQ ID NO: 51 - DNA, 512 bp, Zea mays
  unsure (1)..(512): unsure at all n locations
ctcatgggctgtcnnnnnntnnnnnntnnctnncnnnntcnnannntnnnnnctnnntaatcttgcttacgtacgtatactttacattctataatctactngcaggattacattttgaagaagaaaaactctacaacatatgggnganganagaagaataaactgtagcgcttacatgcatgctcaaaatagcctcataaaccnaatactatctgggatgctagtccagctcctcccatttctgacgtgaatttctggaccgcgtgaagctgtctanttctcttcagatcccgtgccgtttgtctccggaccatcactcctcacagcttgtgctgcattgctcctgtctataataatctgtcacaccaaaactgcaggctttaaaaggcccagacgcattccgggcgcctctacagcccaattaaaaacctcatttcttctctccaggaaagaggnattcnatttcaggacgaacggncacagatgcatggatgcangnnnnnnnnnancna

SEQ ID NO: 52 - DNA, 597 bp, Zea mays
  unsure (1)..(597): unsure at all n locations
tccttgtttcgatcattccaatcgcattctcgacaggagagaactccagagattcagatttgataactggcagacggttcaccaaagcgggaaaggaaccctctgtttggagcactgtncgcctcttccactggtcttctagaccaccttgtgtttttccattttttgtgaatggtgtatcgaaaagaaaacggtcaaaaacacgtgcacgaacagtaccagtagataaagaaaatattctttccctcctgctacccaagtcctcatcctccatcacaggatcaacagcagtgatctgtaaatagcagaagccaggctgcaactcatcagcgttgacttgtctagagtctggaattatgtgtaaagtgtgacttccatctattttagcctcatatatatgactaagcttctccataatatcaccaagacgtacatcccgtggttctctaaatacatactccnttttattgagcttgccaaaacgctcaccatagaaaccaactcggtagtatgtggcatcaatgaatggtataggnctngcctcctgttcaagaattgactcatagatgtttgtgagcgatgtgtggcatttggcca

SEQ ID NO: 53 - DNA, 688 bp, Zea mays
  unsure (1)..(688): unsure at all n locations
agaactttaacttgcttaagggatactgacttactgaattccngagcacaatgctccagatcagcagaagaaagtgagccatgcttattcttcaacccaaacttcatgcagatagtgtnagcagcaaaattaccacnaaagggtgtaacttggccacacgcctggatcttcttggaacaaaatttggtgnaatattctttctggtcttcggcttaggagatgctggtgtcgacaaaatanttgataggggtttggtgaccatctcnataaagttgttagctatgtcncaggtattaacctcctgaagctgaagcttgggccgaatccttctgtgatagactttcagtattttgctagatcnagatcgggagacctgaacagaaggtaatgctggggagtgaagaaccactgcacccacagcaccnaaaccatcaacgcacttctcaatctgaggcattccgaagtcctgagctgacacggtcaaatttcccaacaagtgctcgaccaatgcattagaagaaacaccaggagcctggaaccangcagacttaactgaggcatggacctcaaccgcagcatgaaccggtgtaggagttatnaacgaaatgaatggcgccattactgtagcancagttaaagaacatgccaggatgtcacaattcaaaaaatcnggtgacataactgcagg

SEQ ID NO: 54 - DNA, 634 bp, Zea mays
  unsure (1)..(634): unsure at all n locations
ttattatttggtcgctgatgtgatgaacaactcgtctgcctcaggcttccacgtccgagtggatccgagctggtcacgcatgaggcaaacactccctctcgcgtacnnnnngcagatcatccacctgtccagccccggcgccagccagcacgaccagaccacgctctccctctcgccggtgccgaacactaggcangnngncanncanngtcnnccngncccgcccaaccaaactctccagggatacggctctctctncctctcggtctgtctggtctgcatgtccacatctggagctccacggccacggagatgcttgcggttgcggaagggagctgggctggaggccgcctcgcgttccacacttccgcgaacgtgctgccgcgacgggcgcacgaaagagggacggagaagtgtgccgctcggtccagcccacgcccgctcccccccgcgctataagtagcggcccggtccgccgagtcgagccgtccacagcagagtannntnncacacgcactcgcctcttgtgtctttctttgggagctnnnnncganncacacacagcagcagcagcagcnncaacatacatgcacgctagccnnnnnnnnnnnnnnnnnnnnnnnnnnnnnnnnnt

SEQ ID NO: 55 - DNA, 873 bp, Zea mays
  unsure (1)..(873): unsure at all n locations
cgtgttttccagttgacgttcacggaagtcttgtgtcttttggatagactgatagtgataggcacgcataacagtaaacatattctgtgctccgtgctcatatgancactttaaatctagtgtatacgtgagtaatctagctctgatatcatctcagccttgatagncanncagagagtaaatgaacacggaataagatcccgcataactacctagccacagacactgctgctcctggcgagtggnnnnnnnncgaacaaggtttcccatcttttngngcntgccatacntggattttttttaannnnnnntgtatcnaaattgaanttggaagtgtatactntcccagtcgcacaaagagtgttctttcgacttttgagaagtngnnnnnnnnnnnnnnnnnnnnnnnnnnnnnnnnnnnnnnnnnnnnnnnnnnnnnnnctncntcnntcctaaaataggttngtttagcacttantttttatgtttatattcaaatagatgatgacgaatttacaaatatatatcaaacatatgcattaagtatacattaagtctagtcaacagagagagtaagtattattataatatttgctaaatattttttcataataaacattttttagatacaaaggttgataatacgttttctataaatctagtcaaactaaacaaagtttgacctgaacataaccctaagtgacacttttttggtgtaacgctagctagagcaccaacagcttagcaatgcttgcagtgacatgtttttatttcatttcttttcttgtatccctgttcatcttcaccatttgcacttgttggatgataggcattcaatgatgcagatgtgccttgctagaacatcatgtacta